(19) United States
(12) Patent Application Publication
(10) Pub. No.: US 2024/0138749 A1
(43) Pub. Date: May 2, 2024

(54) METHOD FOR SCREENING OF MOVEMENT DISORDERS

(71) Applicant: Movalytix, Inc., Fort Collins, CO (US)

(72) Inventors: Jay Chandra, Fort Collins, CO (US); Richard Deng, San Ramon, CA (US); Jay Mani Iyer, Baton Rouge, LA (US); Elias Lai, Fort Collins, CO (US); Eryk Pecyna, Fort Collins, CO (US); Zisheng Shang, San Diego, CA (US); Yu Nicole Frances Cabansay, Singapore (SG)

(21) Appl. No.: 18/497,264

(22) Filed: Oct. 30, 2023

Related U.S. Application Data
(60) Provisional application No. 63/420,851, filed on Oct. 31, 2022.

(51) Int. Cl. A61B 5/00; G16H 50/20

(52) U.S. Cl. CPC A61B 5/4082; A61B 5/4836; A61B 5/7264; A61B 5/7275; A61B 5/7475; G16H 50/20; A61B 2560/02; A61B 2560/0431; A61B 2560/0487

(57) ABSTRACT

A method of screening a patient for a degenerative neurological disorder. The method can comprise the steps of: providing a tablet computer having a touchscreen to the patient, instructing the patient to draw a plurality of shapes, wherein each shape of the plurality of shapes is of varying complexity, recording at least one drawing characteristic for each shape drawn by the patient, mathematically analyzing the at least one drawing characteristic to determine an objective rating for each shape, analyzing the objective rating for each shape using a random forest comprising a plurality of decision trees to determine an objective probability that the patient has a neurological deficit, wherein the objective probability that the patient has a neurological deficit is the percentage of the plurality of decision trees which indicate a disease state.

### CROSS REFERENCE TO RELATED APPLICATION

The present application claims the benefit of U.S. Provisional Patent Application Ser. No. 63/420,851 filed on Oct. 31, 2022, titled “METHOD FOR SCREENING OF MOVEMENT DISORDERS”. This reference is incorporated herein in its entirety.

### FIELD

The present disclosure generally relates to a method of screening and diagnosing a patient for neurological deficits. Parkinson's Disease and its variants.

### BACKGROUND

Various neurological disorders have clinically presenting physiological impact on patients. For example, Parkinson's Disease (PD) is an exemplary progressive neurodegenerative disorder of the central nervous system that is pathologically defined by the loss of dopaminergic neurons in the substantia nigra of the brain. PD affects movement, resulting in tremors, rigidity, dyskinesia, and bradykinesia.

PD may result in uncontrollable movements, stiffness, affected gait, issues with balance, issues with coordination, and a general inability to perform fine movements or movements requiring coordinated muscular effort. Furthermore sleep, memory, and behavior can be impacted, often causing or in conjunction with fatigue and/or depression. Disorders related to PD, such as progressive supranuclear palsy or multiple system atrophy are referred to as Parkinsonian syndromes and will often have similar symptoms and diagnostic methodologies.

As it they are degenerative diseases, Parkinsonian syndromes worsen over time and there is currently no known cure. Symptoms are typically managed with medications or a deep brain stimulation procedure

The loss of dopaminergic neurons (the cause of which is unknown) occurs in the basal ganglia, which controls movement, and results in increasing motor impairment.

PD and related disorders are diagnosed by skilled clinicians who evaluate patients in a complex and resource intensive manner. Currently, the best practical methods of diagnosing PD and/or related disorders includes a Datscan (a radiopharmaceutical used in a brain scan) or an MRI (magnetic resonance imaging) scan coupled with a motor assessment by a neurologist.

The neurologist evaluates various qualitative elements, such as eye movement, speech, coordination, reflexes, and other potentially affected traits of Parkinsonian syndromes. The neurologist may administer various cognitive tests and apply a subjective rating of the patient's symptoms according to the Unified Parkinson's Disease Rating Scale (UPDRS).

While comprehensive, the present methodologies rely upon a skilled and trained neurologist in conjunction with subjective ratings which have few discrete or concrete values to diagnose neurological deficits. It is unclear whether a rating by one neurologist is equivalent to an identical rating by a different neurologist.

There is a need therefore, for a cost effective, objective, and rapid screening methodology for determining whether a patient has a neurological deficit, such as a Parkinsonian syndrome, other related disorders that result in loss of motor control, a less severe disorder which mimics these symptoms (such as essential tremor), or none of these types of disorders.

The present disclosure provides a method and apparatus for low-cost, portable, quantitative, and virtually instantaneous screening for neurological deficits.

### SUMMARY OF THE INVENTION

The present disclosure provides a method and apparatus for low-cost, portable, quantitative, and virtually instantaneous screening for neurological deficits.

The novelty of the method and apparatus relates to new shapes being provided to the patient for reproduction, novel measurements and analysis, and the ability to implement an adaptive test, i.e. one in which the difficulty changes for each patient based upon performance. The test can adapt based upon the quality of patient drawings (for example, using a simple distance error to the template drawing measurement) to reduce total drawing time and enhance the efficiency of the test.

The method can comprise the steps of: providing a tablet computer having a touchscreen to the patient, instructing the patient to draw a plurality of shapes, wherein each shape of the plurality of shapes is of varying complexity, recording at least one drawing characteristic for each shape drawn by the patient, mathematically analyzing the at least one drawing characteristic to determine an objective rating for each shape, analyzing the objective rating for each shape using a random forest comprising a plurality of decision trees to determine an objective probability that the patient has a neurological deficit, wherein the objective probability that the patient has a neurological deficit is the percentage of the plurality of decision trees which indicate a disease state, administering a treatment based upon the objective probability that the patient has a neurological deficit, wherein the treatment is specific to the neurological deficit.

### BRIEF DESCRIPTION OF THE DRAWINGS

The detailed description will be better understood in conjunction with the accompanying drawings as follows:

FIG. 1 depicts an example shape to be drawn by a patient.

FIG. 2 depicts an example shape to be drawn by a patient.

FIG. 3 depicts an example shape to be drawn by a patient.

FIG. 4 depicts an example shape to be drawn by a patient.

FIG. 5 depicts an example shape to be drawn by a patient.

FIG. 6 depicts a collection of distance error data.

FIG. 7 depicts azimuth and altitude angle of a writing implement.

The embodiments of the present disclosure are detailed below with reference to the listed Figures.

### DETAILED DESCRIPTION OF THE EMBODIMENTS

Before explaining the present disclosure in detail, it is to be understood that the present disclosure is not limited to the specifics of particular embodiments as described and that it can be practiced, constructed, or carried out in various ways.

While embodiments of the disclosure have been shown and described, modifications thereof can be made by one skilled in the art without departing from the spirit and teachings of the disclosure. The embodiments described herein are exemplary only and are not intended to be limiting.

Specific structural and functional details disclosed herein are not to be interpreted as limiting, but merely as a basis of the claims and as a representative basis for teaching persons having ordinary skill in the art to variously employ the present embodiments. Many variations and modifications of embodiments disclosed herein are possible and are within the scope of the present disclosure.

Where numerical ranges or limitations are expressly stated, such express ranges or limitations should be understood to include iterative ranges or limitations of like magnitude falling within the expressly stated ranges or limitations.

The word “about”, when referring to values, means plus or minus 5% of the stated number.

The use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

When methods are disclosed or discussed, the order of the steps is not intended to be limiting, but merely exemplary unless otherwise stated.

Accordingly, the scope of protection is not limited by the description herein, but is only limited by the claims which follow, encompassing all equivalents of the subject matter of the claims. Each and every claim is hereby incorporated into the specification as an embodiment of the present disclosure. Thus, the claims are a further description and are an addition to the embodiments of the present disclosure.

The inclusion or discussion of a reference is not an admission that it is prior art to the present disclosure, especially any reference that may have a publication date after the priority date of this application. The disclosures of all patents, patent applications, and publications cited herein are hereby incorporated by reference, to the extent they provide background knowledge; or exemplary, procedural or other details supplementary to those set forth herein.

The embodiments of the present disclosure generally relate to a method of screening and diagnosing a patient for neurological deficits, such as Parkinson's Disease and its variants. While Parkinson's disease will be utilized in the following description to help explain the methodology of the present disclosure, it is not the intention to limit this disclosure only to Parkinson's Disease and its variants.

The present disclosure relates to a method of screening a patient for a degenerative neurological disorder. The method can comprise the steps of: providing a tablet computer having a touchscreen to the patient, instructing the patient to draw a plurality of shapes, wherein each shape of the plurality of shapes is of varying complexity, recording at least one drawing characteristic for each shape drawn by the patient, mathematically analyzing the at least one drawing characteristic to determine an objective rating for each shape, analyzing the objective rating for each shape using a random forest comprising a plurality of decision trees to determine an objective probability that the patient has a neurological deficit, wherein the objective probability that the patient has a neurological deficit is the percentage of the plurality of decision trees which indicate a disease state, administering a treatment based upon the objective probability that the patient has a neurological deficit, wherein the treatment is specific to the neurological deficit.

The term “tablet computer” is used broadly to identify any electronic device with a display capable of receiving touch input. This could include a computer and monitor, a laptop, a portable tablet, an iPad®, a smartphone, and the like. Ideally, the tablet computer capable of measuring pressure, speed, variance from path, azimuth angle of a writing implement (such as an included stylus which may or may not communicate electronically with the tablet computer) or finger used to provide touch input. Drawing characteristics can be measured and analyzed for the implementation of the presently disclosed method.

The term “patient” refers to any person being evaluated for a neurological deficit. The patient can be given access to the display capable of receiving touch input. A number of instructions can be provided to the patient. The instructions can be provided on the screen, verbally, in written form, or in combinations thereof.

The patient can be instructed to draw shapes upon the screen. The patient can draw on the screen with a stylus, their finger, or any known input device.

In embodiments, a pattern of the shape can be included on the screen for the patient to trace. In other instances, the patient can be asked to draw freehand. Exemplary shapes include, but are not limited to: a spiral, a spirograph, a rectangle, a circle, an infinity symbol, a rectangular prism, a three dimensional object (such as a cube, a cone, a sphere, and the like), a sinusoid, or any combination thereof. Variations of the shapes can be included as well, such as sinusoids with increasing or decreasing amplitudes, ovals instead of circles, trapezoids instead of rectangles, and the like.

Persons having ordinary skill in the art can determine the appropriate shapes to use for the neurological deficit being evaluated and/or the patient being evaluated. Shapes of increasing, or varying complexity can be selected for the patient to draw. In embodiments, the complexity of a shape can be based upon the patient's performance on the previous shape.

Drawing characteristics can be measured and analyzed for each shape drawn by a patient. For example, a distance error can be calculated for a drawing utilizing distance from drawing coordinates as compared to a template drawing. This distance error can be combined with an evaluation of whether the patient draws through “important” points on the template, such as vertices of shapes, edges of shapes, start points of drawings, end points of drawings, evenly spaced points within the shape, and the like. Persons having ordinary skill in the art can determine which portions of each shape is an “important” point.

The distance error and the evaluation can be used individually, or in conjunction with each other to adaptively administer a test to the patient. Persons having ordinary skill in the art can establish criteria for a “pass” result based upon the patient's performance on a shape in order to provide the patient a more complex shape, or a “fail” result to provide the patient a less complex shape or end the evaluation. The adaptive test can be automated with computer instructions executed by a processor and distance error automatically calculated.

The patient can then be instructed to redraw one or more shapes in a different manner. i.e. the patient may be given a different starting point, told to draw without lifting whatever implement is being utilized off of the screen, told to draw faster, told to draw slower, or other changes as seen fit by persons having ordinary skill in the art.

Various other drawing characteristics can be measured and analyzed. Exemplary characteristics include, but are not limited to: a set of coordinates of the drawing, a pressure applied to the touchscreen for a segment of the drawing, an azimuthal angle of a stylus or a finger used to create the drawing, an altitude angle of the stylus or the finger used to create the drawing, a timestamp associated with each data point, the status of whether stylus or the finger is touching the surface of the screen, the velocity of the stylus movement or the finger movement at a timestamp.

Persons having ordinary skill in the art may generate and utilize a set of coordinates for the patient's drawing for analysis. Alternatively, the patient's drawing can be compared to a standardized set of coordinates for the drawn shape.

Various characteristics can be recorded as a function of time or as a function of location of a portion of the shape. The data can be captured and recorded by the tablet computer, or in embodiments the stylus (or writing implement) can record or transmit the data for recordation. The drawing and shape characteristics and data can be transmitted to a non-transitory data storage medium for storage and/or analysis. This data storage medium can be in electronic communication with the tablet computer.

The characteristics can be chosen by persons having ordinary skill in the art specific to the neurological deficit being tested for. In embodiments, specific combinations of characteristics can be analyzed to determine specific neurological deficits.

The characteristics can be mathematically analyzed to determine an objective rating for each shape drawn by a patient. The objective rating can be numerical, alphanumerical, or combinations thereof.

In embodiments, drawing characteristics can be analyzed to determine at least one of: Radius vs. Theta Regression Sum of Residuals, Radius vs. Theta Regression R2, d2r/dt2 Standard Deviation, dr/dt Standard deviation, Curvature vs. time Regression R2, Rate of Inversion of Pressure, Jerk standard deviation, or Max Jerk.

Radius/Theta measurements are only calculated for shapes with circular components, that is, the circle, spiral, spirograph, and infinity symbol. Please see detailed definitions below:

Radius vs. Theta Regression Sum of Residuals: Error of the radius at various angles as measured in polar coordinates is analyzed.

Radius vs. Theta Regression R2: Error of the radius at various angles as measured in polar coordinates is analyzed.

d2r/dt2 Standard Deviation: First derivative of radius error with respect to time is analyzed.

dr/dt Standard deviation: First derivative of radius error with respect to time is analyzed.

Curvature vs. time Regression R2: Curvature error with respect to time is analyzed.

Rate of Inversion of Pressure: Characteristics of pressure at various points of the drawing are analyzed.

Jerk standard deviation: Spasmodic movements are analyzed.

Max Jerk: Spasmodic movements are analyzed.

Linear Regression-Based Features

In polar coordinates (r,θ), an Archimedean spiral can be described by the equation R(θ)=a+bθ. The coefficient a controls the position of the center of the spiral, and the coefficient b controls the distance between the spiral loops.

When drawing curved trajectories, the velocity is shown to be proportional to the radius of curvature of the trajectory (v=ar where v is the velocity and r is the radius). This relationship also reveals that velocity and time roughly demonstrate a directly proportional relationship, as radius is directly proportional to time in a drawing.

From a physiological standpoint, when a patient draws a spiral from the inside out, pressure tends to increase with the radius of the spiral as the velocity also increases. Utilizing these properties, we apply linear regression to four sets of data: radius vs. theta, velocity vs. radius, velocity vs. time, and pressure (main signal) vs. time. Four quantitative measures of linear regression fits are used as features: the regression coefficient of determination (R-squared values), y-intercept of the regression line, slope of the regression line, and the sum of squared residuals of the regression. The differences be-tween patients and controls in the R-squared and sums of squared residuals metrics may reveal the patients' tendencies to draw with more randomness and less predictability. The regression models were built with a software library's OLS function using default parameters.

Mean & Standard Deviation of the Rates of Change of Radius & Theta

Utilizing the polar properties of the Archimedean spiral, an analysis of the first and second order derivatives of radius as a function of time and theta as a function of time. The radius and theta signals were calculated and then smoothed as described in the data preprocessing section, with radius being the distance from the center of the spiral and theta being the angle from the horizontal polar axis. The derivatives were calculated from the spline fitted functions of these data signals and provide the rate of change at each data point in the smoothed and truncated drawing. The mean and standard deviations of the rate-of-change values provide insight into the stability of the patient' drawing. Patients would be expected to demonstrate higher rates of change (greater mean values) and more variability (higher standard deviation) in the rates of change of their drawings.

Mean & Standard Deviation of the Rate of Change of Radius with Respect to Theta

Another important novel feature is the rate of change of radius with respect to theta (dr/dθ). From the polar equation of the spiral (r=a+bθ), dr/dθ corresponds to the coefficient b in the equation, and a perfect Archimedean spiral should maintain a constant dr/dθ value. Meaningful information can be extracted about the particular drawing's deviation from a perfect spiral through the mean and standard deviation values of the patient's dr/dθ distribution at each point of the drawing. The mean dr/dθ value for healthy controls would be closer to the expected value, and the standard deviation would be lower.

Rate of Inversions

The number and rate of inversions, or the number and rate of changes in directions, have been widely used in Parkinson's handwriting analysis literature. These features are applicable to a range of signals and provide potential indication of loss of fine motor control. The rate of inversions is used in order to normalize the number of inversions collected from signals of drastically different lengths by dividing the raw number of inversions by the time duration of the corresponding drawing. This feature is applied to velocity, acceleration, jerk, pressure, & curvature signals.

A random forest analysis can be performed utilizing the objective ratings as determined above. Persons having ordinary skill in the art can determine a cutoff percentage of decision trees required to indicate a disease state, or a neurological deficit. This cutoff can be adjusted as needed by persons having ordinary skill in the art based upon the patient and/or neurological deficit. This cutoff is also variable based on use case (i.e., specificity vs. sensitivity, specific movement disorder being classified, target false positive rate).

In embodiments, a higher percentage of decision trees indicating a neurological deficit can aid in either determining a severity of the neurological deficit for the patient, a higher confidence that a neurological deficit exists, or a combination thereof. In embodiments, the probability that the patient has a neurological deficit can be compared to a previous probability that the patient has a neurological deficit.

Upon determination that the patient has a neurological deficit, a treatment specific to that deficit is implemented. For example for PD, upon conclusion of testing, if the random forest cutoff is above 55% (classifying an individual as having PD), we recommend more thorough image-based testing (i.e. MRI/DatScan). Further testing can solidify treatment of carbidopa/levodopa to improve/manage motor symptoms.

Further, longitudinal increases in random forest cutoff decision tree percentages may indicate progression of the disease. This can indicate deep-brain stimulation surgery for patients with increased progression as indicated on our drawing test during “the window of opportunity.” This is the window of time in which the individual receives less relief from treatment medications, without the medications being completely ineffective. The present disclosure can be used to implement treatment in conjunction with supporting DatScan, MRI, and MDS-UPDRS images and measurements.

Turning now to the Figures:

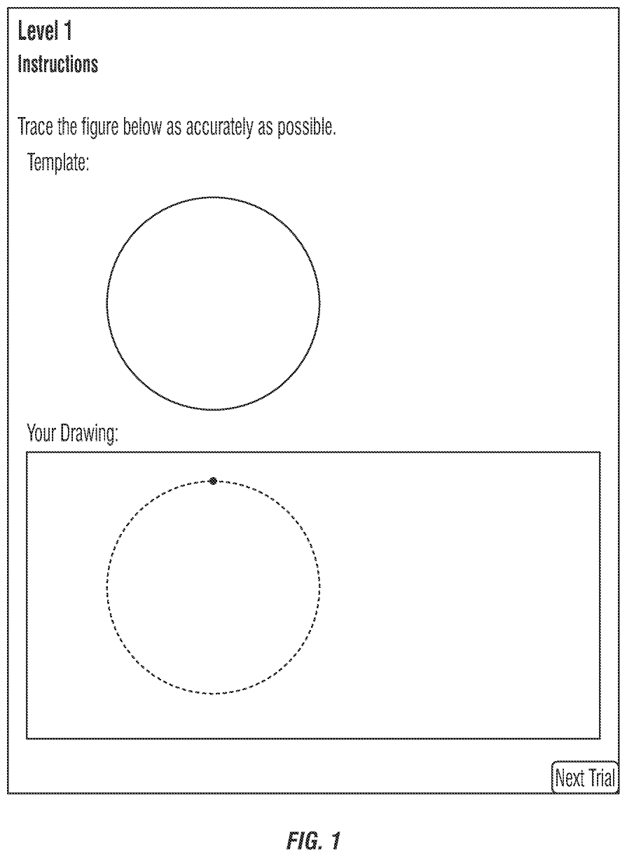

FIG. 1 depicts an example shape to be drawn by a patient.

Herein is shown a simple circle for the patient to trace. Exemplary analyses can include azimuth and altitude angle of a pen (if a pen is used, otherwise characteristics of the position of a finger can be substituted here), curvature of the shape, regression measurements: velocity of drawing on curvature, velocity of drawing on radius, radius vs, theta, frequency space features to identify tremors, and the like.

A custom algorithm can be used to estimate circle dimensions and approximate the isotropy of the circle dimensions. An objective roundness “grade” can be given to this drawing.

On this shape, and any other shape having curvature, a kernel density estimation algorithm to identify multimodality in the data can be used.

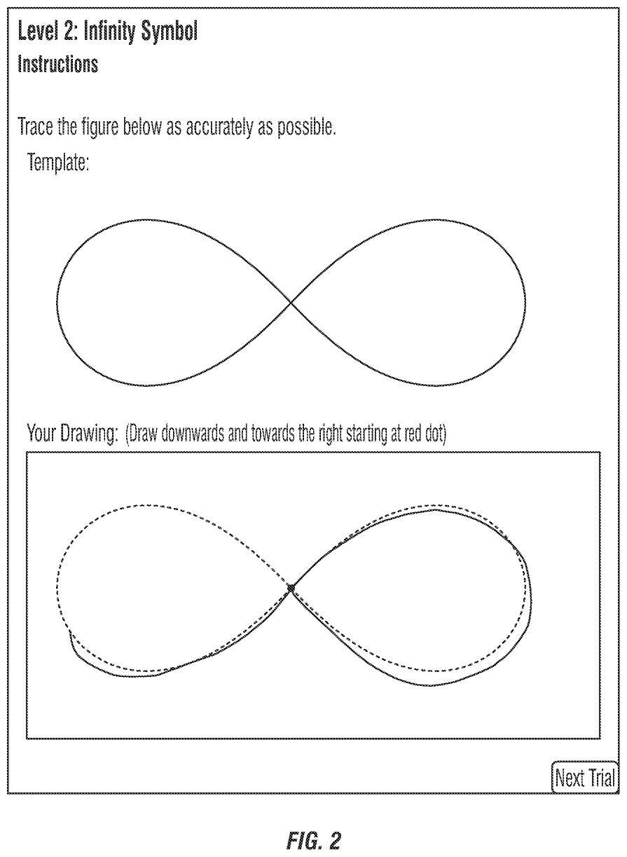

FIG. 2 depicts an example shape to be drawn by a patient.

An infinity symbol is shown here for the patient to trace.

Exemplary analyses here include wobble/stability of pen or finger when drawing, variance of the coordinate path taken, number of pen-pickups on the left and the right side, symmetry of the drawing—i.e. separating the drawing into two halves and comparing standard features of each half. Also, the straightness and Fourier algorithms (discussed below) can be adapted to the infinity symbol to identify wobbly kinematics of the drawing.

The infinity symbol is also a new shape to be utilized in neurological testing as discussed herein, and all of the measured characteristics and calculations are novel to the present method.

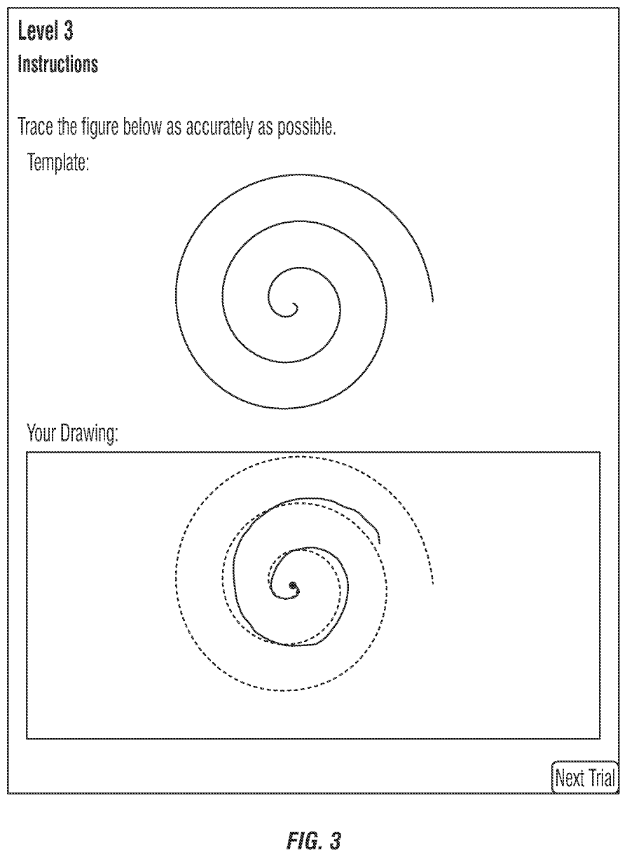

FIG. 3 depicts an example shape to be drawn by a patient.

Shown here is a spiral for the patient to trace.

Exemplary analyses include the analyses described above and the like.

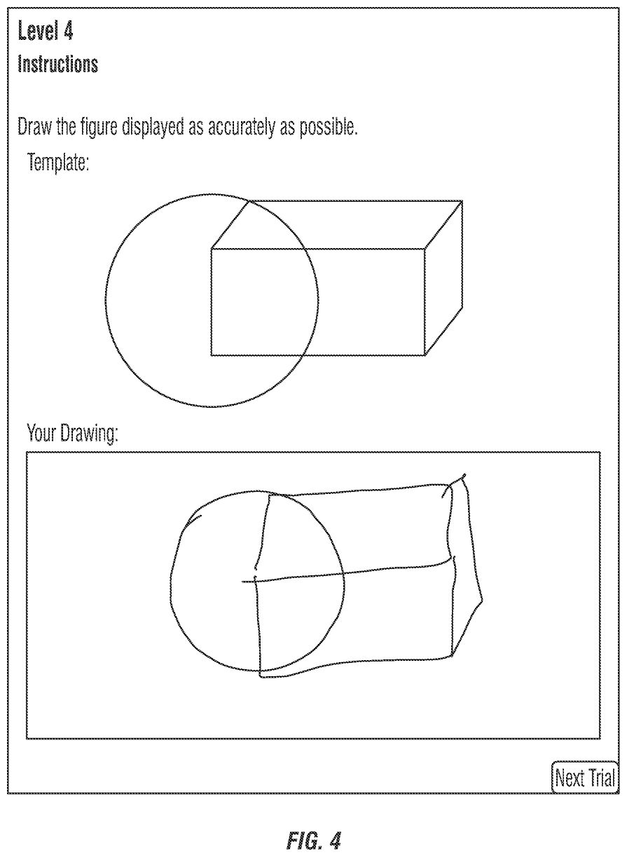

FIG. 4 depicts an example shape to be drawn by a patient.

Shown here is a rectangular prism with circle for the patient to trace. This is also a new shape to be utilized in neurological testing as discussed herein.

Exemplary analyses include accuracy of straight edges of rectangular prism, roundness of circle, ability to draw the 3D image, bimodal distribution of curvature, and the like. In embodiments, a local linear fitting algorithm can be used over small regions of fixed time and the average fit scores can be used as a metric for straightness. A tuned Fourier Decomposition-based algorithm can be to assess line straightness on a micro scale.

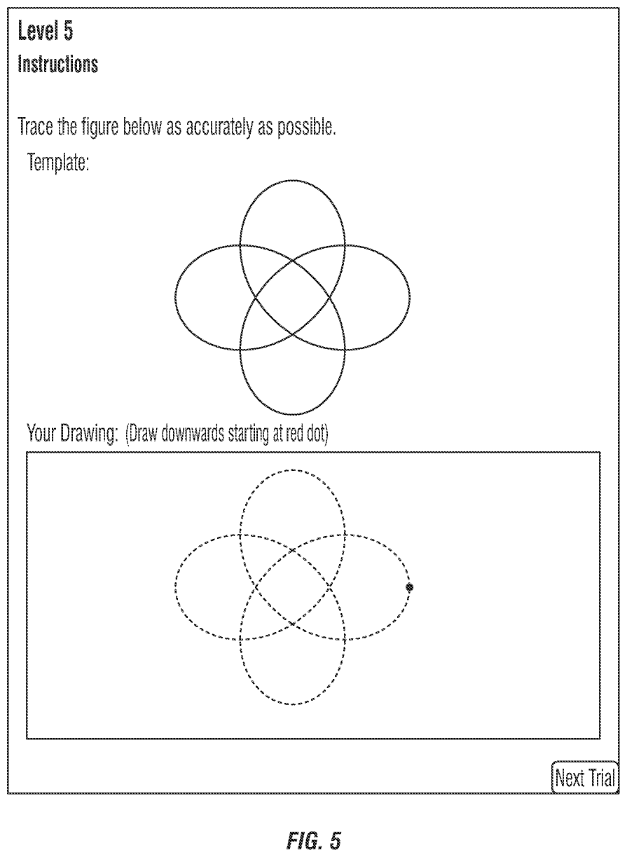

FIG. 5 depicts an example shape to be drawn by a patient.

Shown here is a spirograph for the patient to trace. This is also a new shape to be utilized in neurological testing as discussed herein.

Exemplary analyses include the analyses described above. Also novel to the present method is analyzing each “petal” of the spirograph and comparing characteristics to each other.

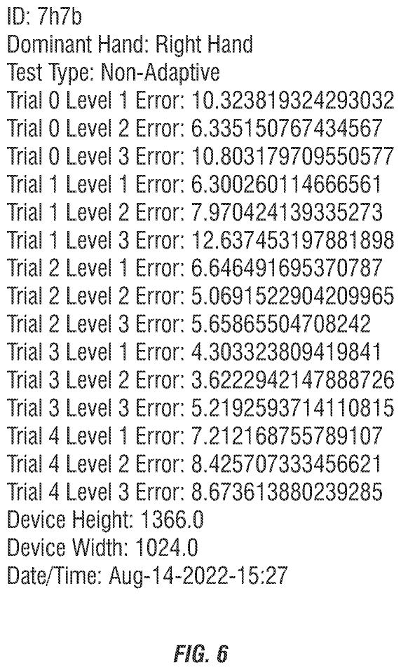

FIG. 6 depicts a collection of distance error data.

Data such as this can be analyzed and a patient score can be generated by persons having ordinary skill in the art for each patient.

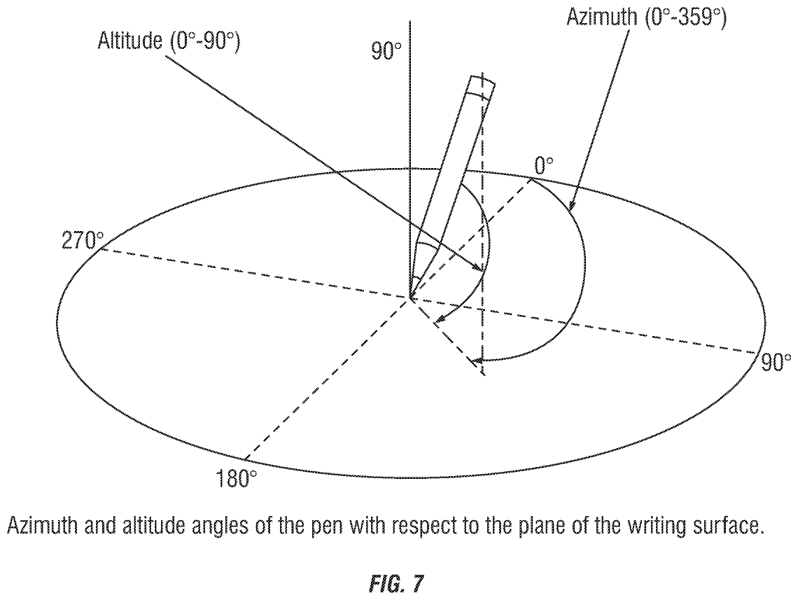

FIG. 7 depicts azimuth and altitude angle of a writing implement.

Various data can be collected from a writing implement if used for patient testing.

Exemplary data to be analyzed can include azimuth angle, attitude angle, number of pick ups, speed of drawing, pressure of drawing, and the like.

While the present disclosure emphasizes the presented embodiments and Figures, it should be understood that within the scope of the appended claims, the disclosure might be embodied other than as specifically enabled herein.

What is claimed is:

1. A method of screening a patient for a degenerative neurological disorder, comprising
a. providing a tablet computer having a touchscreen to the patient;
b. instructing the patient to draw a plurality of shapes, wherein each shape of the plurality of shapes is of varying complexity;
c. recording at least one drawing characteristic for each shape drawn by the patient;
d. mathematically analyzing the at least one drawing characteristic to determine an objective rating for each shape;
e. analyzing the objective rating for each shape using a random forest comprising a plurality of decision trees to determine an objective probability that the patient has a neurological deficit, wherein the objective probability that the patient has a neurological deficit is a function of the percentage of the plurality of decision trees which indicate a disease state; and
f. administering a treatment based upon the objective probability that the patient has a neurological deficit, wherein the treatment is specific to the neurological deficit.

2. The method of claim 1, further comprising the step of determining a severity of the neurological deficit if the objective probability that patient has a neurological deficit is greater than a cutoff value.

3. The method of claim 1, wherein at least one shape of the plurality of shapes is:
a. a spiral;
b. a spirograph;
c. a rectangle;
d. a circle;
e. an infinity symbol;
f. a rectangular prism;
g. a three-dimensional object, wherein the three-dimensional object is generalized for projected surfaces and curved surfaces;
h. a sinusoid; or
i. a combination thereof.

4. The method of claim 1, further comprising the step of: determining a most complex shape which the patient is able to draw by administering an adaptive test.

5. The method of claim 4, further comprising the step of: instructing the patient to draw the most complex shape in a different manner.

6. The method of claim 1, wherein the drawing characteristics comprise at least one of:
a. a set of coordinates of the drawing;
b. a pressure applied to the touchscreen for a segment of the drawing or at a timestamp of the drawing;
c. an azimuthal angle of a stylus or a finger used to create the drawing for a segment of the drawing or at a timestamp of the drawing;
d. an altitude angle of the stylus or the finger used to create the drawing for a segment of the drawing or at a timestamp of the drawing;
e. a timestamp associated with each point of the drawing;
f. the status of whether stylus or the finger is touching the surface of the screen for a segment of the drawing or at a timestamp of the drawing; or
g. the velocity of the stylus movement or the finger movement at a timestamp for a segment of the drawing or at a timestamp of the drawing.

7. The method of claim 1, further comprising the step of comparing the probability that the patient has a neurological deficit to a previous probability that the patient has a neurological deficit.

8. The method of claim 1, wherein the tablet computer comprises a stylus.

9. The method of claim 6, wherein the drawing characteristics are analyzed to determine at least one of:
a. Radius vs. Theta Regression Sum of Residuals;
b. Radius vs. Theta Regression R²;
c. d²r/dt² Standard Deviation;
d. dr/dt Standard deviation
e. Curvature vs. time Regression R²;
f. Rate of Inversion of Pressure;
g. Jerk standard deviation; or
h. Max Jerk.
